(19) United States
(12) Patent Application Publication
(10) Pub. No.: US 2026/0356881 A1
(43) Pub. Date: Nov. 12, 2026

(54) SUPRAMOLECULAR PROTECTION OF ACIDIC AMINO ACID SIDE CHAINS USING PILLAR[6]ARENE DERIVATIVES IN PEPTIDE SYNTHESIS

(71) Applicant: Yong Ma, San Pablo, CA (US)

(72) Inventor: Yong Ma, San Pablo, CA (US)

(21) Appl. No.: 19/714,276

(22) Filed: Jun. 19, 2026

Related U.S. Application Data
(62) Division of application No. 18/915,273, filed on Oct. 14, 2024.

(51) Int. Cl. C07K 1/06; C07K 1/00

(52) U.S. Cl. CPC C07K 1/063; C07K 1/006

(57) ABSTRACT

The invention relates to supramolecular protection of acidic amino acid side chains during peptide synthesis. Protected amino acid compositions are provided comprising glutamic acid, aspartic acid, or derivatives thereof associated with pillar[6]arene dodeca-methylamine through non-covalent interactions including hydrogen bonding. The resulting protected amino acid derivatives are useful as building blocks in peptide synthesis, including solid-phase peptide synthesis. Methods are provided for preparing the protected amino acid compositions by combining an acidic amino acid and pillar[6]arene dodeca-methylamine in an aqueous solution and reacting the resulting complex with an amino-protecting reagent. Methods are further provided for incorporating the protected amino acid compositions into peptide chains and subsequently removing the pillar[6]arene dodeca-methylamine protective molecule following peptide synthesis. The disclosed technology reduces reliance on conventional covalent side-chain protecting groups and may reduce the consumption of organic solvents and chemical reagents during peptide manufacturing.

### CROSS-REFERENCE TO RELATED APPLICATIONS

The present application is a divisional application of U.S. patent application Ser. No. 18/915,273, filed Oct. 14, 2024, entitled “Protection of Side Chains of Amino Acids by Host Guest Affinity Molecules in Peptide Synthesis,” currently pending. The entire disclosure of U.S. patent application Ser. No. 18/915,273 is incorporated herein by reference in its entirety.

### FIELD OF THE INVENTION

The present invention relates to the protection of side chains of amino acids in peptide synthesis. Particularly, the present invention involves a method for creating side chain-protected amino acids using host-guest affinity molecules for solid-phase peptide synthesis and encompasses compositions of protected side chains of Lysine (Lys), Aspartic Acid (Asp), and Glutamic Acid (Glu) achieved through host-guest affinity molecules for facilitating solid-phase peptide synthesis. Using host-guest affinity molecules to protect side chains of Lys, Asp and Glu offers advantages for contributing to a greener planet by minimizing chemical reactions and chemical solvent usage. Additionally, the cost of side chain protection is dramatically reduced.

### BACKGROUND OF THE INVENTION

Conventional peptide synthesis commonly employs covalent side-chain protecting groups for acidic amino acids such as glutamic acid and aspartic acid. Such protecting groups typically require chemical attachment and subsequent removal using strong acidic reagents and substantial quantities of organic solvents.

The present invention provides a supramolecular alternative utilizing host-guest affinity interactions between acidic amino acid side chains and pillar[6]arene derivative molecules. The resulting complexes function as protected amino acid building blocks suitable for peptide synthesis while reducing the need for conventional covalent protection chemistry.

The amino acid Lys has two amino groups: an α-amino group and an ε-amino group. Typically, α-amino group is protected by Fmoc and ε-amino group is protected by Boc or Alloc.

Traditionally Boc (tert-Butyloxycarbonyl) Group is be used to protect side chain of Lys, Boc is stable under acidic conditions of peptide coupling and can be removed by treatment with strong acids like trifluoroacetic acid (TFA). The removal requires strong acidic conditions, which might be detrimental to peptides sensitive to acids.

The side chain protection employed in peptide synthesis for Lys also can use Alloc (allyloxycarbonyl). Alloc safeguards the side chain amino group of lysine by forming a bond with it. Utilization of the allyloxycarbonyl (Alloc) group to protect side chain of Lys, typically, is accomplished by reacting Lys with allyl chloroformate in the presence of a base, such as triethylamine (TEA), in an organic solvent such as dichloromethane (DCM) or tetrahydrofuran (THF). This reaction produces the Lys derivative with the Alloc group linked to the amino group. Following the completion of peptide synthesis, the Alloc protecting group is removed to expose the free amino group of Lys. Deprotection is usually achieved through treatment with PhSiH3.

For Asp and Glu, the common side-chain protecting group used is the tert-butyl ester (t-Bu). Protecting the side chain of Asp/Glu (Asp or Glu) is commonly achieved by shielding the carboxyl group with the t-Bu protecting group. This attachment of the t-Bu protecting group to the carboxyl group of Asp/Glu occurs through a chemical bond formed in an organic solvent. Upon completion of peptide synthesis, the t-Bu protecting group is removed using 90% TFA, revealing the free carboxyl group of Asp/Glu.

In peptide synthesis, safeguarding the side chains of amino acids involves several considerations: Orthogonal Protecting Groups: utilizing protecting groups that can be selectively removed under particular conditions without affecting other protected functional groups is essential. This facilitates stepwise deprotection and sequential peptide elongation. Temporary Modifications: Certain protecting groups are introduced as temporary modifications during peptide synthesis and are later removed after the desired chemical reactions have occurred. Protecting Group Stability: It's crucial to choose protecting groups that remain stable under the reaction conditions. However, they should also be easily removable under mild conditions to avoid damaging the peptide.

We have developed a novel approach to enhance environmental friendliness and streamline side chain protection of amino acids in peptide synthesis. We utilize molecules 18-crown-6(18C6) and Pillar[6]arene(P6A) derivative as protective agents for the side chain of Lys and side chains of Asp/Glu during peptide synthesis to improve environmental friendliness and also streamline the side chain protection process in peptide synthesis.

In solid-phase peptide synthesis (SPPS), Fmoc-AAs (where Fmoc stands for fluorenylmethoxycarbonyl and AAs for amino acids) are the most commonly used building blocks for sequential peptide elongation. We have developed procedures for preparing Fmoc-Lys with side chains protected by 18C6, abbreviated as Fmoc-Lys(18C6), and for utilizing Fmoc-Lys(18C6) as building blocks in SPPS.

Additionally, we have devised a procedure for synthesizing a derivative molecule of Pillar[6]arene(P6A) derivative with additional 12 methylamine group, that is Pillar[6]arene dodeca-methylamine abbreviated as P[6]A-DMA. These 12 secondary amino groups are strategically designed to enhance affinity towards carboxyl groups on the side chains of Asp and Glu. We utilize the molecule P[6]A-DMA as a protective group to shield carboxyl groups on the side chains of Fmoc-Asp and Fmoc-Glu, abbreviated as Fmoc-Asp(P[6]A-DMA) and Fmoc-Glu(P[6]A-DMA) respectively. Both Fmoc-Asp(P[6]A-DMA) and Fmoc-Glu(P[6]A-DMA) are used for as building blocks in SPPS.

18C6 is utilized to protect the side chain of Lysine, while P[6]A-DMA is employed for protecting the side chains of Aspartic Acid (Asp) and Glutamic Acid (Glu) during peptide synthesis. 18C6 and P[6]A-DMA molecules feature cavities at their centers. These cavity structures exhibit host-guest affinity with certain side chain groups of amino acids. The 18C6 molecules exhibit a unique and distinctive host-guest affinity towards the amino group of the side chain Lys and 18C6 is utilized for protecting the Lys side chain during peptide synthesis.

The molecule P[6]A-DMA exhibits a distinct affinity for the carboxyl groups on side chairs of Asp and Glu, and is utilized for protecting the Asp and Glu side chains during peptide synthesis.

Both 18C6 and P[6]A-DMA molecules bind to Lys and Asp/Glu side chains through hydrogen bonds, eliminating the need for chemical reactions to attach protective molecules to the side chain groups of Lys and Asp/Glu. This simplifies the side chain protection procedure significantly, reducing the consumption of organic solvents. Consequently, this procedure offers advantages for contributing to a greener planet by minimizing chemical reactions and solvent usage. Additionally, the cost of side chain protection is dramatically reduced.

### SUMMARY OF THE INVENTION

In one aspect, the invention provides protected acidic amino acid compositions comprising glutamic acid or aspartic acid associated with pillar[6]arene dodeca-methylamine through hydrogen bonding.

In another aspect, the invention provides Fmoc-protected glutamic acid and Fmoc-protected aspartic acid compositions associated with pillar[6]arene dodeca-methylamine.

In another aspect, the invention provides methods for synthesizing such protected amino acid compositions.

Study of 18C6 and P6A have garnered significant attention lately due to their enhanced affinity towards the side chains of amino acids. Several published articles have discussed new developments in these molecules, particularly their affinity towards the side chains of Lysine and Asp/Glu through cavity-guest interactions. 18C6 and P6A possess cavity structures, and recent studies have elucidated how these cavities interact specifically with the side chains of amino acids. Professors Steed and Pease have provided comprehensive summaries on the topic. 18C6 molecules are known for their robust binding to protonated Lysine and their distinctive affinity for potassium cations. FIG. 1 shows molecule structure of 18-Crown-6 (18C6).

Other researches have unveiled the strong affinity of Pillar[6] towards the carboxyl group of the side chains Asp/Glu. Pillar[6]arenes represent elegant macrocyclic systems known for their potential as host systems across various applications, including ion and molecule sensing. Investigating the binding interactions between Pillar[6]arenes and amino acids provides valuable insights into host-guest chemistry and molecular recognition. Several review articles and research papers delve into this intriguing field. One notable article is by Professor Qunpeng Duan, published in Tetrahedron Letters. It discusses the synthesis of a water-soluble pillar[6]arene dodecaamine and its specific binding affinity towards the side chains of Asp/Glu in aqueous environments. FIG. 2 shows basic structure of Molecule P6A (Pillar[6]arenes).

Fmoc-AA, where the alpha amino groups of amino acids are protected by Fmoc, is one of the most widely used building blocks in solid-phase peptide synthesis. Taking advantage of the specific affinity of 18C6 and P6A towards Lys and Asp/Glu, respectively, we developed a procedure for making molecule of Fmoc-Lys with side chain protected by 18C6 denoted as Fmoc-Lys(18C6), FIG. 3 shows molecule structure of Fmoc-Lys(18C6).

Pillar[6]arene dodeca-methylamine abbreviated as P[6]A-DMA is synthesized as a protective group to protect carboxyl group on side chain of Glu. In order to making Pillar[6]arenes soluble in aqueous solution and having NH group to form hydrogen bond with carboxyl groups of Glu, we modified Pillar[6]arenes by substituting the twelve Br(Bromine) in Pillar[6]arenes with twelve-NHCH3, abbreviated as P[6]A-DMA. We also developed another procedure to synthesize Fmoc-Glu with the side chain protected by P[6]A-DMA, denoted as Fmoc-Glu(P6A-DMA). FIG. 4 shows molecule structures of Fmoc-Glu(P6A-DMA.

The Fmoc-Lys(18C6) and Fmoc-Glu(P6A-DMA) compounds can serve as building blocks in solid-phase peptide synthesis.

### BRIEF DESCRIPTION OF THE DRAWINGS

FIG. 1. molecule structure of 18-Crown-6 (18C6).

FIG. 2. basic structure of Molecule P6A (Pillar[6]arenes)

FIG. 3. molecule structure of Fmoc-Lys(18C6).

FIG. 4 molecule structures of Fmoc-Glu(P6A-DMA).

FIG. 5. the chemical reaction for synthesis of Fmoc-Lys(18C6)

FIG. 6 Flow chart for making Fmoc-Lys(8C6)

FIG. 7 Chemical reaction for preparing P6A

FIG. 8, making derivative of P6A with 12 methylamine groups (Pillar[6]arene dodeca-methylamine (abbreviated as P[6]A-DMA)) for better affinity toward carboxyl groups of Asp/Glu

FIG 9, Fmoc-Asp/Glu with P6A-DMA to form molecule Fmoc-Glu(P6A-DMA)

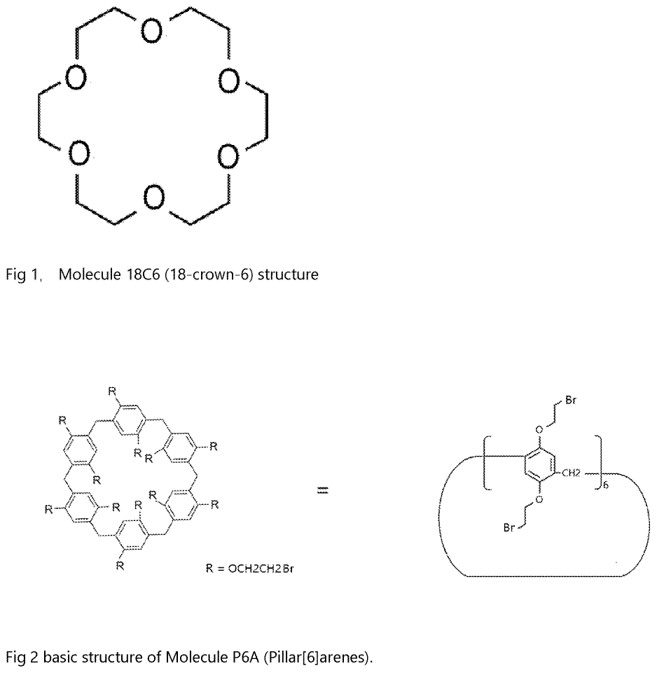

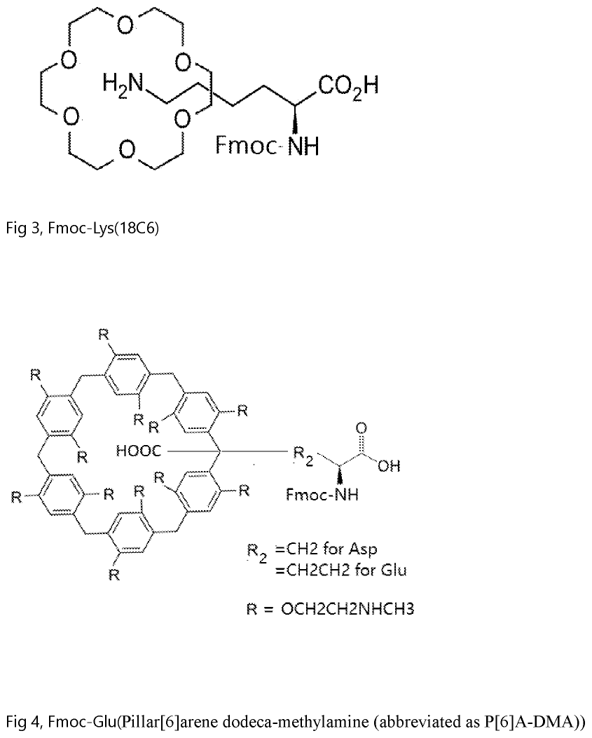

All illustrations of the drawings are for the purpose of describing selected versions of the present invention and are not intended to limit the scope of the present invention.

### DETAILED DESCRIPTION OF THE INVENTION

### Preparing Fmoc-Lys(18C6)

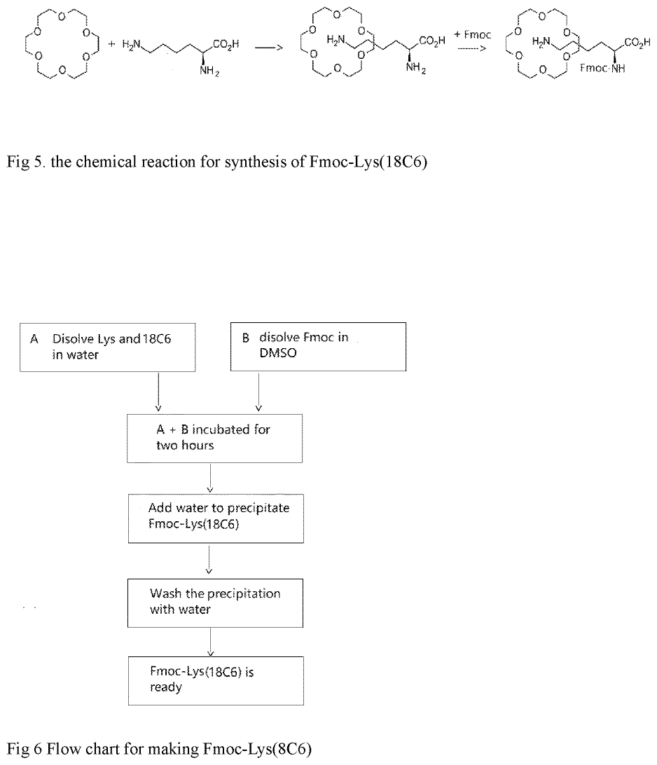

Initially, Lys is incubated with 18C6 in an aqueous solution to ensure that 18C6 binds to the Lys side chain. Following this, the Lys-18C6 complex in the aqueous solution is introduced into a DMSO (dimethyl sulfoxide) solution containing Fmoc. This leads to the formation of Fmoc-Lys(18C6) in the solution. FIG. 5 illustrates the chemical reaction involved in the synthesis of Fmoc-Lys(18C6)

Procedure for Synthesis of Fmoc-Lys with Side Chain Protected Molecule 18C6
1. Dissolve Lysine (Lys) and 18C6 in water to prepare Solution A.
2. Dissolve Fmoc in DMSO to prepare Solution B.
3. Slowly add Solution A dropwise into Solution B while stirring. Allow the reaction to proceed for 2 hours.
4. The product, Fmoc-Lys(18C 6), will form in the reaction mixture.
5. To remove unreacted Lys and 18C6, add an additional amount of water to the mixture. Lys and 18C6 are highly soluble in water, whereas Fmoc-Lys(18C6) has poor solubility in water, leading to its precipitation. The precipitate will mainly consist of Fmoc-Lys(18C6).
6. Filter the precipitate to isolate Fmoc-Lys(18C6), which is now ready for peptide synthesis.

The flowchart for the synthesis of Fmoc-Lys(18C6) is shown in FIG. 6. 18C6 can be removed using Potassium ions or amines after peptide synthesis is completed.

### Preparing Pillar[6]arene dodeca-methylamine (Abbreviated as P[6]A-DMA)

### Synthesis of P6A and P[6]A-DMA

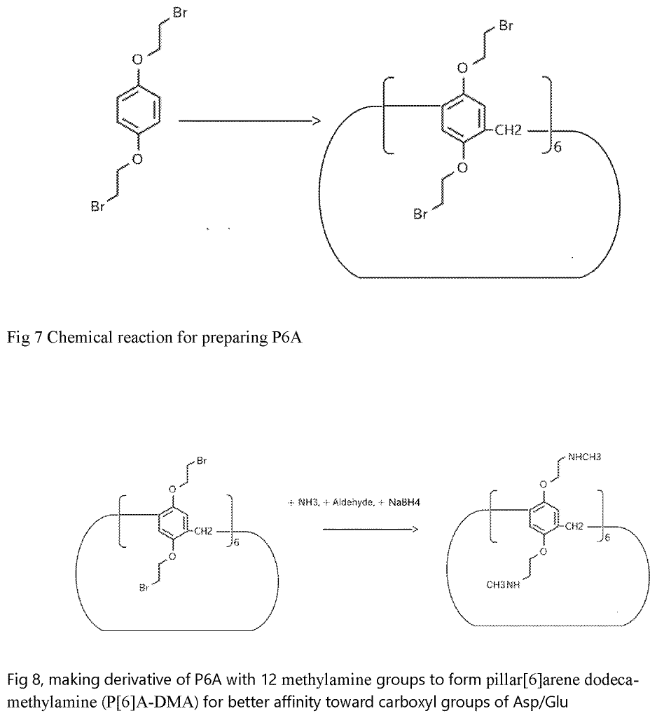

Pillar[6]arene (P6A) is typically synthesized using 1,4-bis(2-bromoethoxy)benzene and paraformaldehyde (—(CH₂O—)n) in the presence of BF₃·OEt₂ as a catalyst, with chlorohexane as the solvent. This reaction is illustrated in FIG. 7.

To make P6A soluble in aqueous solutions and introduce NH groups capable of forming hydrogen bonds with the carboxyl groups of Asp or Glu, we modified P6A by substituting the Br groups with NH₃. Subsequently, the resulting —NH₂ groups were deactivated via the addition of an aldehyde to form imine groups. These imine groups were then reduced using NaBH₄, yielding P[6]A-DMA, This process is illustrated in FIG. 8.

### Preparing Fmoc-Glu With Side Chain Protection by P[6]A-DMA

A. Glutamic acid (Glu) and pillar[6]arene dodeca-methylamine are dissolved in an aqueous solution to form Solution A, with a molar ratio of Glu to pillar[6]arene dodeca-methylamine in the range of 1:1.1.
B. Fmoc-Cl is dissolved in DMSO to form Solution B, with a molar ratio of Fmoc-Cl to Glu in the range of 1.1:1.
C. Solution A is slowly added to Solution B to form the reaction mixture.
D. The reaction mixture is kept at room temperature for 60 minutes, during which the Glu derivative is formed.

Wherein said glutamic acid derivative molecule, the protective group, pillar[6]arene dodeca-methylamine, in the glutamic acid derivative molecule, can be removed after the completion of solid-phase peptide synthesis. This removal can be achieved using 0.1%-0.5% of a dicarboxylic acid, such as glutaric acid or adipic acid.

P[6]A-DMA binds to the Fmoc side chain through hydrogen bonding in the aqueous solution, without the need for a chemical reaction. The process can be represented as:

P[6]A-DMA+Fmoc-Glu→Fmoc-Glu(P[6]A-DMA)

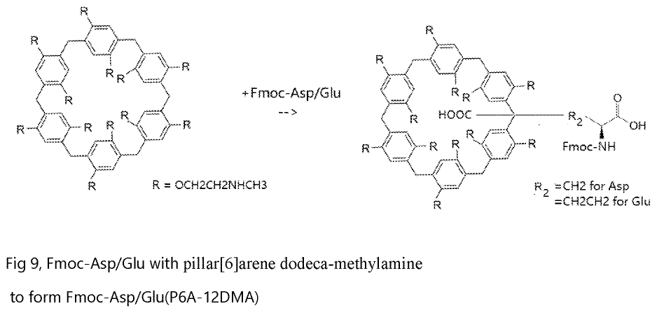

FIG. 9 illustrates this process. After peptide synthesis is complete, P[6]A-DMA can be removed using a carboxylic acid treatment.

### Example Experiment for Preparation of Fmoc-Lys(18C6)

1. Preparation of Solution A:
   Dissolve approximately 1 gram of Lysine (Lys) and 1.8 grams of 18C6(18 -crown-6 ether) in 20 mL of water. Incubate for 2 hours, as 18C6 is slow to dissolve.
2. Preparation of Solution B:
   Dissolve 1.7 grams of Fmoc (9-Fluorenylmethoxycarbonyl chloride) in 80 mL of DMSO (Dimethyl sulfoxide).
3. Mixing Solutions:
   Add Solution A dropwise into Solution B while stirring. Allow the mixture to react for 2 hours.
4. Formation of Fmoc-Lys(18C6):
   The reaction results in the formation of Fmoc-Lys(18C6), where the side chain of Lysine is protected by the crown ether (18C6). The Fmoc group selectively reacts with the alpha-amino group of Lys, leaving the side chain amine protected by 18C6.
5. Purification by Solubility:
   Purify the Fmoc-Lys(18C6) based on its hydrophobic properties. While Lys and 18C6 are highly soluble in water, Fmoc-Lys(18C6) is poorly soluble.
6. Precipitation:
   Add 200 mL of water to the reaction mixture. Precipitation of Fmoc-Lys(18C6) occurs, as it is poorly soluble in water, while Lys and 18C6 remain in solution.
7. Filtration and Yield:
   Filter the precipitate, which contains the Fmoc-Lys(18C6). The resulting product is now ready for use in peptide synthesis. The yield is approximately 55%.

### Chemical Reactions:

1. Lys+18C6→Lys(18C6) (due to the affinity of 18C6 for the side chain amine of Lys)
2. Lys(18C6)+Fmoc→Fmoc-Lys(18C6) (since the 18C6 protects the side chain amine, Fmoc reacts only with the alpha-amino group of Lys).

This procedure yields Fmoc-Lys(18C6), where the side chain is protected by 18C6, making it suitable for peptide synthesis.

### Example Experiment: Synthesis of Taurine-Lys(18C6) Peptide Using Fmoc-Lys(18C6) as a Building Block

1. Prepare Taurine-Anion Exchange Resin:

Incubate 1 gram of taurine with 5 grams of an anion exchange resin in 20 mL of water. Wash to remove excess taurine.
2. Dissolve Fmoc-Lys(18C6):
   Dissolve 0.5 Grams of Fmoc-Lys(18C6) in 4 mL of DMSO.
   Add 2 grams of Tween 80 to the mixture.
3. Introduce to Resin:
   Add the Fmoc-Lys(18C6) solution to the taurine-bound anion exchange resin in 20 mL of water.
4. Add Coupling Agent:
   Introduce 1-Ethyl-3-(3-dimethylaminopropyl)carbodiimide (EDC) as the coupling agent.
5. Adjust pH:
   Adjust the pH to 4.5-5.0 and allow the reaction to proceed for 60 minutes.
6. Formation of Dipeptide (Taurine-Fmoc-Lys(18C6)):

The dipeptide Taurine-Fmoc-Lys(18C6) should now be formed.
7. Wash:
   Wash the resin with a 10% Tween solution to remove excess EDC.
8. Deprotect Fmoc Group:
   Remove the Fmoc protective group by adjusting the pH to 9.5-10.
9. Final Wash:
   Wash the resin again with a 10% Tween solution.
10. Result:
   The dipeptide Taurine-Lys(18C6) should now be bound to the resin.

### Deprotection of Protective Group 18C6

1. Add 2 mL of a 1% KCl solution.
2. Taurine-Lys(18C6)+KCl→Taurine-Lys+K18C6. Potassium has a stronger affinity for 18C6 than the lysine side chain, leading to the removal of 18C6 from the lysine side chain.
3. A 20% salt solution can be used to elute the dipeptide from the anion exchange resin.

The chemical reactions are as follows:
Taurine+Anion Exchange Resin→Resin-Taurine (binding through ionic interaction in water)
Resin-Taurine+Fmoc-Lys(18C6)+EDC→Resin-Taurine-(Fmoc)-Lys(18C6) (coupling reaction facilitated by EDC)
Taurine-(Fmoc)-Lys(18C6) at pH 9.5-10→Resin-Taurine-Lys(18C6)+Fmoc (deprotection of Fmoc at pH 9.5-10)
Resin-Taurine-Lys(18C6)+KCl→Resin-Taurine-Lys (removal of 18C6 by K⁺ ions)
Resin-Taurine-Lys+20% salt→Resin+Taurine-Lys (elution of the dipeptide with 20% salt solution)

1. A protected acidic amino acid composition comprising:
(a) an acidic amino acid selected from glutamic acid, aspartic acid, and derivatives thereof; and
(b) a pillar[6]arene dodeca-methylamine molecule associated with a side-chain carboxyl group of the acidic amino acid through hydrogen bonding,
wherein the composition is suitable for use as a building block in peptide synthesis.

2. The composition of claim 1, wherein the acidic amino acid comprises Fmoc-protected glutamic acid or Fmoc-protected aspartic acid.

3. A method of preparing a protected acidic amino acid composition comprising:
(a) dissolving an acidic amino acid and pillar[6]arene dodeca-methylamine in an aqueous solution;
(b) dissolving an amino-protecting reagent in a water-miscible organic solvent;
(c) combining the solutions to form a reaction mixture; and
(d) recovering a protected acidic amino acid composition.

4. A method of peptide synthesis comprising:
(a) providing the protected acidic amino acid composition of claim 1;
(b) incorporating the protected acidic amino acid composition into a peptide chain during peptide synthesis; and
(c) removing the pillar[6]arene dodeca-methylamine following peptide synthesis.
